(19) United States
(12) Patent Application Publication
(10) Pub. No.: US 2008/0097226 A1
(43) Pub. Date: Apr. 24, 2008

(54) Evaluation of cardiac function using left ventricular pressure during LVAD support

(76) Inventor: Patrick I. McConnell, Salt Lake City, UT (US)

Correspondence Address: ROETZEL & ANDRESS, 1375 EAST 9TH STREET, CLEVELAND, OH, 44114, US

(21) Appl. No.: 11/450,611

(22) Filed: Jun. 9, 2006

Related U.S. Application Data
(63) Continuation-in-part of application No. 11/150,855, filed on Jun. 9, 2005, now abandoned.

(51) Int. Cl. A61B 5/02; A61N 1/362

(52) U.S. Cl. 600/485; 600/16; 600/526

(57) ABSTRACT

A method and apparatus for determination of cardiac function by monitoring left ventricular (LV) pressure and varying ventricular assist device (VAD) speed, utilizes a relationship of the end-diastolic LV pressure (LVEDP) to an estimate of LV work calculated from the LV pressure signal by the triple product (TP): dp/dtmax*HR*LVSP, wherein the slope of a regression analysis of the comparison of TP vs LVEDP is indicative of a patient's cardiac efficiency and analogous and comparable to preload recruitable stroke work as calculated from direct volume measurement of the LV, and of native cardiac function of a patient supported by a VAD, and related control systems for a VAD for controlling operation of the VAD according to the method.

### RELATED APPLICATIONS

This application is a continuation-in-part of U.S. application Ser. No. 11/150,855, filed Jun. 9, 2005.

### BACKGROUND OF THE INVENTION

The following abbreviations are used in this specification.

### Abbreviations:

bpm beats per minute
EDP end-diastolic pressure (LV)
Eₑₛ end-systolic elastance (slope ESPVR)
ESPVR end-systolic pressure volume relationship
dP/dtₘₐₓ maximum derivative of LV pressure
dP/dtₘᵢₙ minimum derivative of LV pressure
LV Left ventricle or ventricular
LVAD Left ventricular assist device
MAP Mean arterial pressure
M_{TP} Slope TP-EDP
M_{w} Slope PRSW
PRSW preload recruitable stroke work
SW Stroke Work (LV)
TP Triple product (LV) Tau—Weiss method

In clinical studies, a telemetered left ventricular (LV) pressure manometer (LVP1000®: Transoma Medical, St Paul, Minn.) has been placed transmyocardially to monitor LV pressures during left ventricular assist device (LVAD) support. This or similar technology could potentially improve the care of patients who have both a LV pressure monitor and an assist device. Left ventricular volume unloading with an LVAD may permit the recovery of cardiac function and remodeling sufficient for device explant—so-called ‘Bridge to Recovery’. Unfortunately, the reality of this therapeutic approach is that very few patients, likely less than 5% of chronic heart failure patients supported with a LVAD, demonstrate enough cardiac function for LVAD removal. However, there is agreement within the scientific community that with better diagnostic capabilities to direct device operation or concomitant therapeutics, better success rates could be realized. Including the relevant recommendations by the Working Group on Recovery from Heart Failure with Circulatory Assist of the National Heart, Lung and Blood Institute Reinlib and Abraham. JCardFail 2003 9: 459-631 for the serial determination of anatomical structure and functional parameters aimed at the proper assessment of recovery, for markers and predictive factors (hemodynamics) of ‘recoverable’ hearts to be identified and for the design of mechanical assist devices and systems specifically for cardiac recovery.

If less arduous methods existed for evaluating LV function during LVAD support, permitting greater frequency or even automated assessments, it is likely that improvements in device operation and ‘weaning’ strategies could be realized allowing for better ‘Bridge to Recovery’ therapy. A reduction in adverse events and morbidity associated with ‘Destination’ therapies could also potentially be realized with chronic LV hemodynamic monitoring—by allowing the operation of devices at more appropriate support levels, device wear and some potentially negative effects to the native heart (e.g. right heart failure, arrhythmias, etc.) as well as the patient (e.g. thromboembolic events) could be reduced or avoided. To date, no relevant or specific methods exist for determining cardiac function from the LV pressure signal alone in patients supported with an axial flow LVAD.

The ability to quantify systolic myocardial performance is essential for the development of strategies to effectively utilize left ventricular assist devices to ‘Bridge to Recovery’ and also to potentially improve strategies aimed at reducing morbidity for patients ‘Bridged’ to either ‘Transplantation’ or ‘Destination’ therapies. If the currently poor success rate for ‘Bridge to Recovery’ therapy in chronic heart failure patients is a valid metric, then the current methods of functional, metabolic, histological and molecular assessment has, unfortunately, proved to be of little value in improving ‘Bridging’ strategies. Of particular importance, and beyond the mere ability to quantify systolic performance, is the need for frequent and reliable assessments of cardiac function both in the context of directing concomitant therapy and also for the institution of a closed-loop feedback mechanism between sensor and device.

Ferrari and colleagues recently reported on monitoring patients' load-independent cardiac function using pressure-volume (P-V) analysis derived from the offline analysis of catheter acquired LV pressure signals and echocardiographically-derived LV volumes at implant and explant of an axial flow LVAD. In these cases, the establishment of the end-systolic pressure volume relationship (ESPVR) was performed in a novel way by using the LVAD to acutely unload the LV—establishing the P-V relationship. Yet, theoretical and technical issues related with the interpretation of endsystolic elastance or the ESPVR during axial-flow unloading limit the interpretation of end-systolic elastance under axial flow unloading conditions. For example, as the LVAD unloads the LV, the systemic circulation is supported by limiting changes in mean arterial pressure and the LV end-systolic pressure despite large changes in LV volume. A situation has then been created where changes in the end-systolic pressure are not dependent on changes in LV volume—compromising any index reliant on the coupling of these particular factors (i.e. ESPVR).

### Axial Flow LV-Unloading and Pressure-Volume Analysis

The ESPVR (slope: Eₑₛ) as an index of cardiac function is reliant on the coupling of LV end-systolic pressure with end-systolic volume, a correlation directly compromised by axial flow LV unloading. Axial flow supports the systemic circulation and essentially preserves the end-systolic pressures (FIGS. 2A and 2B) until LV volume is insufficient to allow LV ejection (LVSP<MAP, FIG. 1). Thus, a situation is created where the changes in the end-systolic pressure are not dependent on changes in LV volume (i.e. ESPVR), making the Eₑₛ a poor estimate of cardiac function when varying volume with a continuous flow LVAD.

Like the ESPVR, the preload-recruitable stroke work (PRSW), relies on P-V analysis to provide a load-independent index of cardiac function. However, unlike the ESPVR, the slope of the PRSW (M_{w}) remains sensitive to cardiac functional status during axial flow LV-unloading because both LV SW and end-diastolic volume (EDV) vary dependently with the degree of axial flow support. Moreover, the PRSW is linear over a wider range of LV volumes than the ESPVR [Takaoka H. Suga H. Goto Y. Hata K. Takeuchi M. Cardiodynamic conditions for the linearity of the preload recruitable stroke work. Heart and Vessels. 1995: 10(2): 57-68.1, a situation we have recently demonstrated for LV unloading with an axial flow LVAD. Unfortunately, the greatest challenge with P-V derived indices of cardiac function is the complexity of repetitively measuring LV volumes in LVAD supported patients.

Emerging technology will soon allow for the chronic assessment of telemetered left ventricular (LV) pressure in patients supported with LVADs; however, specific methods have not been developed for the use of LV pressure during LVAD support. In the future, LV pressure-derived parameters could improve the outcomes of patients supported with mechanical assist devices.

### SUMMARY OF THE INVENTION

The present disclosure is of a method of calculating an estimated work performed by a beating heart and varying the speed of a blood pump as a function of the estimated work, wherein calculating the estimated work involves the steps of detecting a heart rate (HR) of the beating heart; monitoring left ventricular (LV) pressure in the beating heart; calculating a maximum derivative of the LV pressure (dp/dt max); determining a left ventricular systolic pressure (LVSP) from the monitored LV pressure; and calculating a triple product (TP) defined by the equation: TP=dp/dt max*HR*LVSP, and the speed of the blood pump is varied as a function of the estimated work by a blood pump controller.

The present disclosure is further of a method and apparatus for determination of cardiac function by monitoring left ventricular (LV) pressure and varying ventricular assist device (VAD) speed, utilizes a relationship of the end-diastolic LV pressure (LVEDP) to an estimate of LV work calculated from the LV pressure signal by the triple product (TP): dp/dtmax*HR*LVSP, wherein the slope of a regression analysis of the comparison of TP vs LVEDP is indicative of a patient's cardiac efficiency and analogous and comparable to preload recruitable stroke work as calculated from direct volume measurement of the LV, and of native cardiac function of a patient supported by a VAD, and related control systems for a VAD for controlling operation of the VAD according to the method.

The present disclosure is further of an apparatus which includes a blood pump; a controller operatively coupled to the blood pump; a pressure sensor providing left ventricular (LV) pressure values to the controller; a heart rate sensor providing heart rate (HR) values to the controller; and a controller operative to receive the sensed LV pressure values, receive the sensed HR values, calculate an estimated work, and deliver control signals to the blood pump in response to the estimated work.

These and other aspects of the disclosure are further described in detail herein with reference to the accompanying Figures and Tables, such descriptions and examples being representative of the principles and concepts of the disclosure and not limiting to the scope of the claims to the same.

### DESCRIPTION OF THE FIGURES

FIG. 1 illustrates representative hemodynamic tracings during a LVAD unloading ‘run’;

FIGS. 2A and 2B illustrate resulting set of P-V loops before and after esmolol;

FIGS. 2C and 2D illustrate the PRSW and TP/EDP relationships before and after esmolol administration, respectively;

FIGS. 3A-3D illustrate LVAD outflow graft blood flow versus LVAD speed (3A) and individual parameters (3B-3D) of the lef betricular triple product during a baseline run and after emolol relative to LVAD blood flow;

FIG. 4 illustrates in linear regression a high degree of correlation between TP and SW during LVAD unloading ‘run’ and expressed by the equation y=1.0S6x+448.3, R²=0.86; P<0.001 before and by the equation y 0.814x+175.6, R²=0.764; P<0.001 after esmolol, and

FIG. 5 is a plot of M_{TP} against LVDEP for various PE infusions.

FIG. 6 is a Table of data on hemodynamics during LV unloading “run” with and axial-flow LVAD before and after β1-adrenergic blockade.

FIG. 7 is a Table of data on response after autonomic blockade to phenylephrine (PE) infusion in a single animal.

FIG. 8 is a schematic diagram illustrating a system in accordance with one exemplary embodiment of the present invention.

FIG. 9 is a schematic diagram showing a system in accordance with an additional exemplary embodiment of the present invention.

FIG. 10 is a schematic diagram showing a system in accordance with another exemplary embodiment of the present invention.

### DETAILED DESCRIPTION OF PREFERRED AND ALTERNATE EMBODIMENTS

A method and apparatus is disclosed to assess cardiac function during axial-flow LVAD support that would allow for a) the frequent and b) the repetitive assessment of LV function from c) a single hemodynamic source—telemetered LV pressure. The relationship of the LV triple product (TP: LVSP*dP/dtₘₐₓ*HR) to LVEDP, TP/EDP (slope: M_{TP}), is used to provide an index sensitive to changes in cardiac function like the preload-recruitable stroke work (PRSW). A comparison of TP/EDP to PRSW in axial-flow LVAD subjects is made before and after beta-adrenergic blockade with esmolol.

### LVAD Placement and Instrumentation

In accordance with this disclosure, adult Suffolk sheep (N=6, 78±3 Kg) underwent placement of axial-flow LVAD. Each animal was instrumented with a telemetered LV pressure manometer, an outflow graft transit time flow probe, and endocardial LV long and short axis piezoelectric crystals to derive LV volume and stroke work (SW). In unsedated sheep, LV load was varied by increasing LVAD speed from 6,000 to 10,880±120 rpm (“run”). During ‘run’, the PRSW (slope: Mw), and simultaneously, the relationship of LV triple product (TP) to LV end-diastolic pressure (TP/EDP, slope: M_{TP}) were determined by least squares regression analyses before and after 31 blockade (esmolol 5 mg/kg/mm). Comparisons were made using One-way ANOVA and multiple linear regression analysis. VAD support, 4.5±0.31 L/min, was maintained for >72 hrs prior to study. When VAD speed was increased from 6,000 rpm (‘run’), the LVAD flow (QV) increased (1.2±0.25 to 5.8±0.71 L/min) while the LV SW (3061±747 to 1556±410 mmHg*mL), LV TP (3127±397 to 1019±335×105⁵) and LV EDP (18.2±1.2 to 9.7±1.8 mm Hg) decreased (P<0.0 1). The relationships of TP-EDP and the PRSW established during LVAD ‘run’ were sensitive and reduced by esmolol administration (M_{TP}: 158±23.8 to 71±15.1; P<0.001 and Mw: 117±15.8 to 72±9.4; P<0.001). Like the PRSW, the relationship of TP to EDP established during LVAD unloading ‘run’ was sensitive to changes in cardiac function after esmolol administration.

The adult sheep (N=6, 78±3 Kg) underwent placement of an axial-flow LVAD (Heart Mate II®, Thoratec Corp., Pleasanton, Calif.) through a left thoracotomy while avoiding cardiopulmonary bypass. The LVAD inflow cannula was positioned through the LV apex and the outflow graft (16 mm) was sewn to the descending thoracic aorta. The pump remained within the thorax and the transcutaneous power cable was tunneled to the animals' left flank.

Fluid-filled catheters (Tygon®) were secured with suture into the descending thoracic aorta and left atrial appendage. In four of six animals (N4), two pairs of piezoelectric crystals (2 mm, Sonometrics Inc., New London, Ontario Canada) were surgically placed endocardially in the equatorial plane at the mid papillary level (short axis, SA), and anteriorly at the LV base and near the LV apex (long-axis, LA) for calculation of LV volumes. Telemetered manometers (TL1 1M3-D70-PCP, Data Sciences International, St. Paul, Minn.) were secured within the right ventricle (RV, N=5) and the LV chambers. An ultrasonic transit-time flow probe (16 mm, Transonic Inc., Ithaca, N.Y.) was placed around the LVAD outflow graft. All catheters and cables were exited from between the animals' scapula. Prior to performing studies, animals were allowed to recover typically for at least 1 week while the LVAD was operated continuously at approximately 9,000 rpm (partial support).

Aortic and left atrial fluid filled catheters were connected to calibrated Statham pressure transducers (Model: P23XL; Biggo-Spectramed, Ocknard, Calif.) and amplified (Gould, Valley, Ohio) for their respective pressures. The telemetered pressure waveforms were acquired via UA-10 receiver (DSI, St Paul, IVIN) and electronically calibrated while adjusting for atmospheric conditions and the accuracy of LV pressure was confirmed against calibrated aortic and left atrial pressure signals. Sonomicrometer signals were analyzed for cardiac-cycle dependent (end-diastolic and end-systolic) and waveform dependent (minimum, maximum, mean etc) parameters. The signals from the outflow graft flow probe were amplified and electronically calibrated before each experiment. All waveforms were collected (at 1 kHz) and analyzed by a 16-channel data acquisition and software system (IOX, version 1.7, EMKA Technologies, Falls Church Va.). Hemodynamic waveforms were analyzed (IOX) and averaged data (2 second) was output to tab delimited files and accessed using a standard spreadsheet software program.

Left ventricular volume was calculated in real-time from endocardial positioned sonomicrometers using the equation: (SA²*LAπ/6)*1000 (ml). Left ventricular triple product (TP) was calculated on a per beat basis within software (IOX) from the telemetered LV pressure signal using the following equation: LVSP*dPdtₘₐₓ*HR—where LVSP was the LV systolic pressure, dPdtₘₐₓ was the maximal derivative of LV pressure and HR was the heart rate. The LV stroke work (SW) was also calculated (∫LVP*dLV volume) in real-time within software (IOX) on a per beat basis.

Baseline LVAD supported data (‘on support’) was collected from awake, unrestrained and standing animals while the LVAD support was continued up to 10,000 rpm. LV unloading with the axial flow LVAD was performed after the pump speed was reduced to 6,000 rpm and the animals' hemodynamics were allowed to stabilize for up to 2 minutes at this speed. Then the LVAD was programmed to “run” up to a point where the LVSP<MAP or approximately 11,000 rpm (100 rpm/second). In each case, the TP/EDP relationship and the PRSW were derived from the same run.

The responses of TP/EDP and PRSW to changes in inotropy were evaluated after β1-adrenergic blockade with esmolol hydrochloride. On the same day as the baseline ‘run’, animals were administered an intravenous bolus of esmolol (25 mg) followed by intravenous esmolol infusion (5 mg/kg/mm). A ‘run’ was repeated after at least 1 minute of esmolol infusion.

The effect of increased afterload on the TP/EDP relationship was assessed in a single animal on three separate days. Prior to each study, autonomic blockade was produced with atropine (0.1 mg/kg i.v.) and metoprolol (5 mg i.v.) to prevent baroreflex activation, during PE infusion. Phenylephrine was infused at 0.01, 0.1 and 0.25 mcg/kg/min with the goal to increase LV systolic pressure by approximately 10, 20 and 30 mm Hg, respectively.

Data is expressed as the mean±SEM. Data was collected during a single experimental period or day; therefore, comparisons of hemodynamic after autonomic blockade and PE doses as well as data between time points: ‘on support’ and 6,000 rpm, 6,000 rpm and 11,000 rpm within groups and 6,000 rpm before and after esmolol were made using a one-way ANOVA with repeated measures design (SigmaStat 2.03, Systat Software Inc., Point Richmond, Calif.). If the F-ratio was found to exceed a critical value <0.05) the post hoc Bonferroni's method was applied to perform pair-wise comparisons. The slopes of the PRSW (M_{w}) and the TP/EDP (M_{TP}) relationships were derived from least squares linear regression analysis of plots (2-second averages) for the SW versus the end-diastolic volume and for the TP versus the LVEDP, respectively. Multiple linear regression analysis was used to compare M_{TP} and M_{w} before and after esmolol infusion and to compare MTP after autonomic blockade and PE infusion.

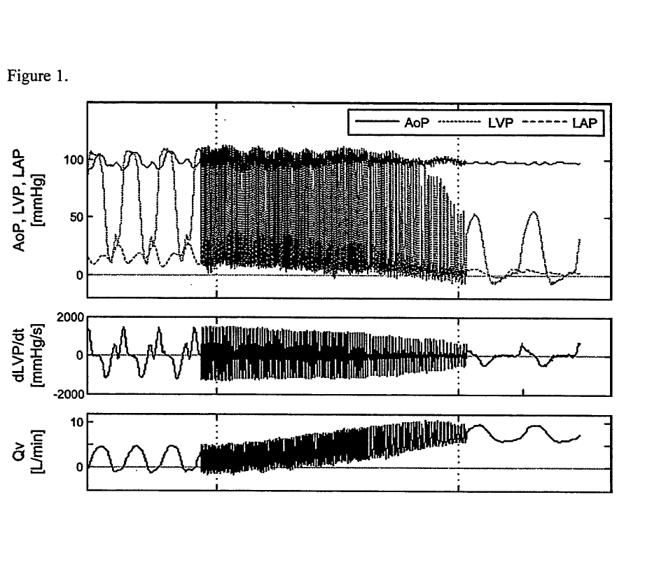

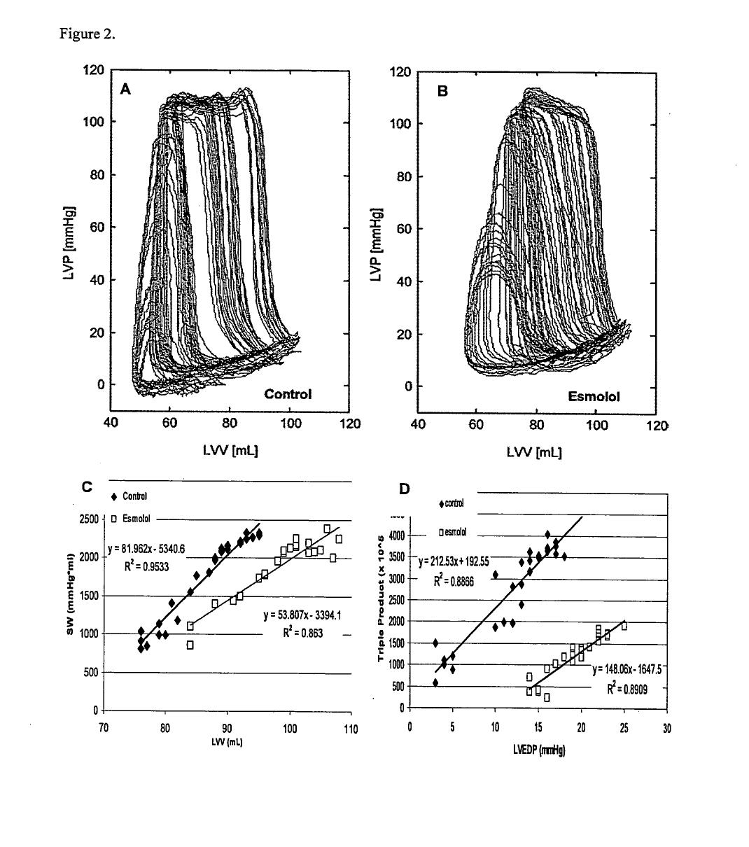

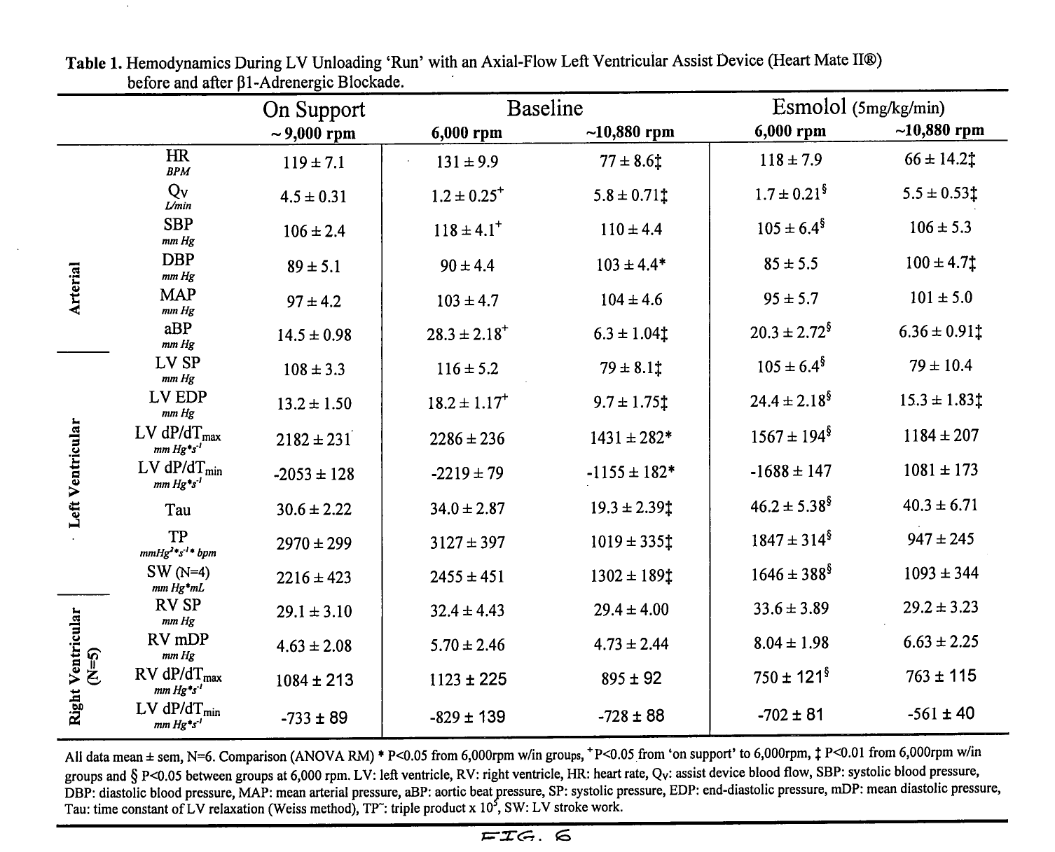

Six animals were studied after LVAD implantation and instrumentation. Animals were partially supported with the LVAD on average for 13 days (range 3 to 40 days). A typical ‘Run’ progressed from 6,000 rpm to 10,880±120 rpm. Representative hemodynamic tracings during LVAD unloading ‘run’ are shown in FIG. 1 with a resulting set of P-V loops before and after esmolol in FIGS. 2A and 2B. The PRSW and TP/EDP relationships before and after esmolol are shown in FIGS. 2C and 2D, respectively. Esmolol reduced M_{TP} from 159±23.8 to 71±15.1 mm Hg*s⁻¹*bpm (N=6; P<0.001) and M_{w} from 117±15.8 to 72±9.4 mm Hg (N=4; P<0.001). Right ventricular dP/dtₘₐₓ was reduced after esmolol; otherwise, all other RV hemodynamics were not significantly altered by LV reloading and subsequent LVAD unloading ‘run’. Additional, hemodynamic data from ‘runs’ before and after esmolol infusion are presented in Table 1, FIG. 6.

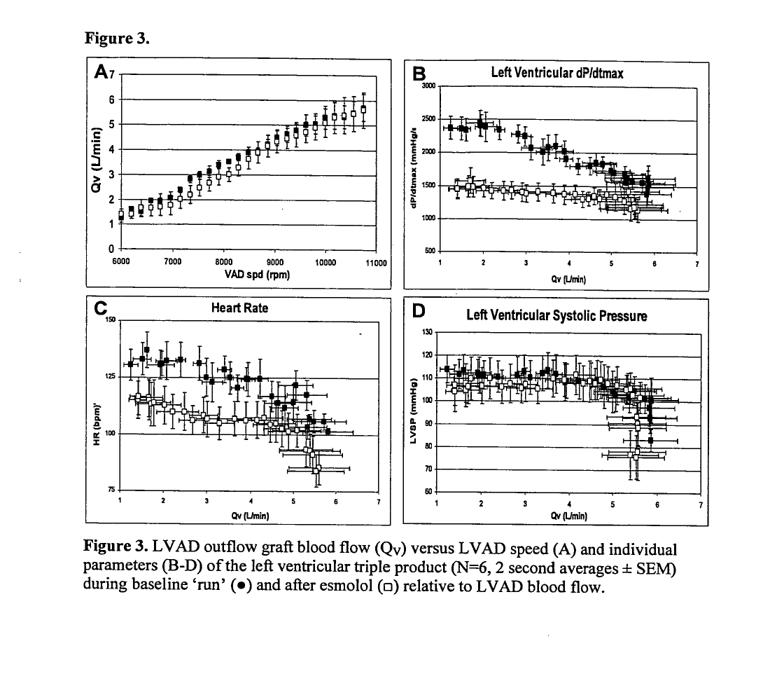

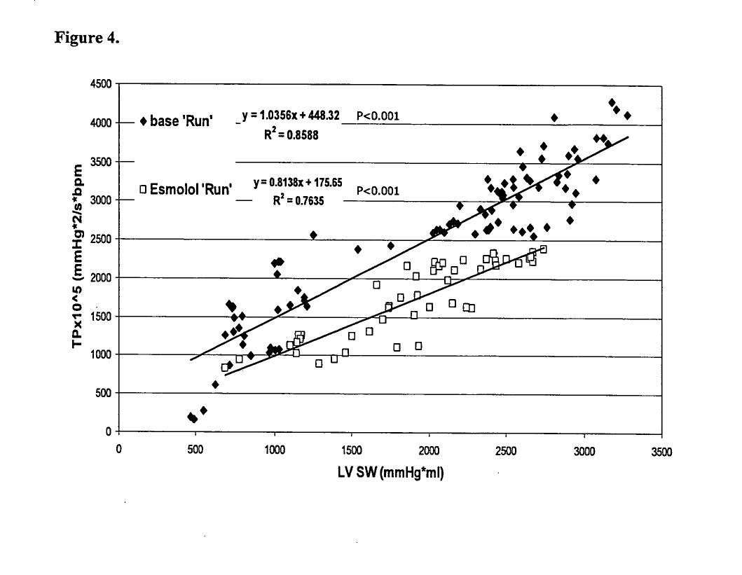

Outflow graft blood flows (Qᵥ) were nearly identical before and after esmolol (FIG. 3A). LVAD blood flow increased linearly with LVAD speed until plateau. Each component of the LV TP (dP/dtₘₐₓ, LVSP, and HR) relative to Qᵥ during ‘run’ is shown in FIGS. 3B, 3C and 3D. The predominant effect of esmolol on the TP/EDP slope was reduced LV contraction velocity (dP/dTₘₐₓ, FIG. 3B) and, although HR was lower after esmolol, the change in HR was not appreciably different after esmolol (FIG. 3C). Left ventricular systolic pressure, in FIG. 3D, was not observed to be significantly reduced until late in the ‘run’. In FIG. 4, linear regression demonstrated a high degree of correlation between TP and SW during LVAD unloading ‘run’ and was expressed by the equation y=1.086x+448.3, R²=0.86; P<0.001 before and by the equation y 0.814x+175.6, R²=0.764; P<0.001 after esmolol.

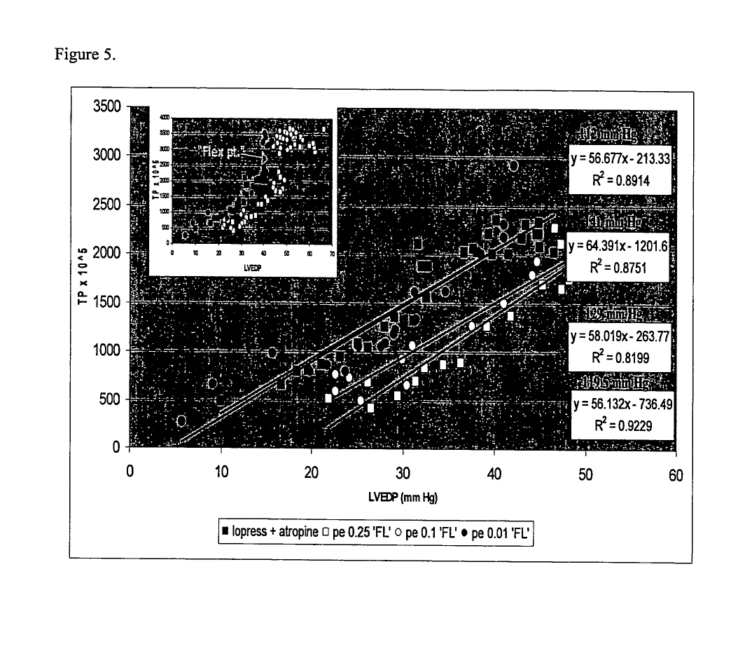

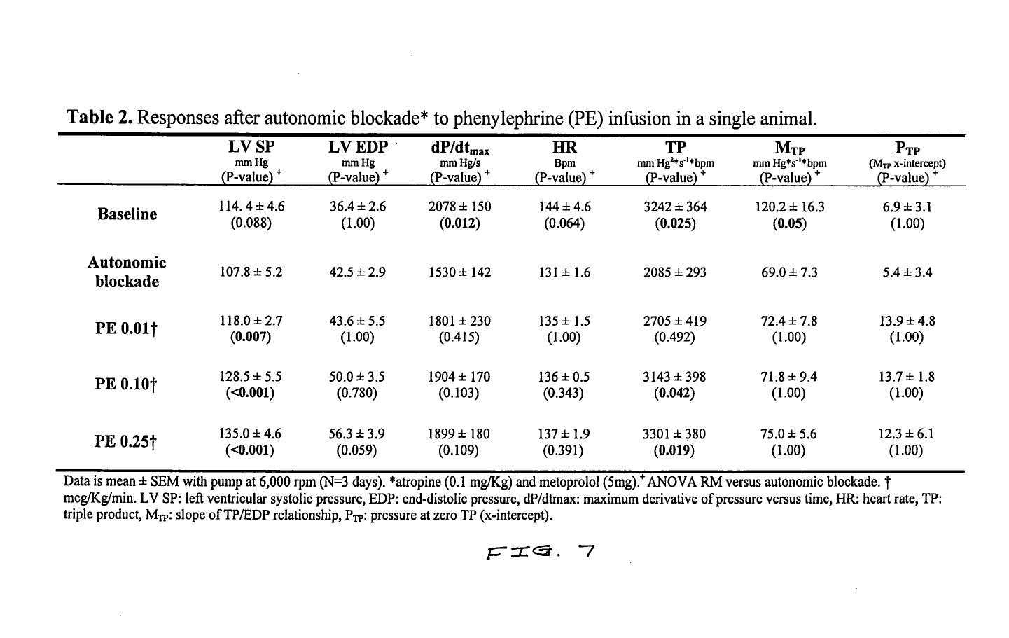

Selected data after autonomic blockade and PE infusion are presented in Table 2, FIG. 7. On three separate days in a single animal, infusion of 0.01, 0.1 and 0.25 mcg/kg/min of PE after autonomic blockade increased LVSP by 10.2±2.56, 20.7±1.51 and 27.2±0.93 mm Hg, respectively (P<0.007). Heart rate was 144±4.6 bpm at baseline (pump speed 6,000), 146+5.1 bpm after atropine and 131±1.6 bpm after atropine and metoprolol (P=0.064 vs. baseline; power 0.45, N=3). In FIG. 5, a cluster of points or plateau was noted at higher filling pressures associated with PE infusions (inset), below which, the M_{TP} was observed to be linear. We have defined the position that the M_{TP} resumes a linear relationship as its “flex point” (FIG. 5—inset). There was no appreciable difference in M_{TP} below the ‘flex point’ for each dose of PE (afterload) from that of complete autonomic blockade; even though, Mrp was reduced with autonomic blockade (Table 2, FIG. 7 and FIG. 5). In this same animal, a plateau [glower et al 1985] was not observed in the TP/EDP relationship either before or after complete autonomic blockade, or on a separate day after esmolol alone (e.g. in FIG. 2). Accounting for PE dose and day, the variability in MTP was 4.59±0.68 (7.0±1.17%) despite the higher variability observed in the TP: 673±92.6 (33.4±4.7%).

### Left Ventricular Triple Product and the TP/EDP

The LV TP as defined herein provides a surrogate of SW derived from the LV pressure signal by accounting for pressure, heart rate and contractility (i.e. dP/dtₘₐₓ). The dP/dtₘₐₓ or the velocity of LV contraction is traditionally known to be a poor measure of intrinsic cardiac contractility because of its reliance on the LV developed pressure, thereby making it preload and afterload dependent in addition to being heart rate dependent. Several studies have demonstrated that estimates of myocardial work that rely on LV dP/dtₘₐₓ typically correlate poorly with myocardial oxygen consumption (MVO₂). However, the observation that changes in the TP and resulting TP/EDP correlate with changes in the SW and resulting PRSW (respectively) is intriguing because it appears to be inconsistent with others studies. The reasons for the linear correlation between TP and SW are believed to be specific to the method used for LV unloading (i.e. LVAD) in this report.

As illustrated by FIGS. 1 and 3B, left ventricular dP/dtₘₐₓ was progressively and linearly reduced during LV unloading with an axial flow LVAD, an observation not apparent with vena cava occlusions (data not shown). Vena cava occlusions quickly reduce the LV developed pressure that reduces the LV dP/dtₘₐₓ nonlinearly and, therefore, confounds the interpretation of dP/dtₘₐₓ as a measure of ‘intrinsic contractility’. As previously stated, when reducing LV preload with a continuous flow LVAD, the LV systolic and developed pressures are relatively well preserved (FIGS. 1 and 3D) because the systemic circulation is supported by the LVAD—a very different event from vena cava occlusion. Therefore, changes in dP/dtₘₐₓ are likely more reflective of meaningful changes in preload affecting the contractile state of the myocyte (preload recruitable function) and also possibly minimizing reflex activation.

A further consideration of the estimation of cardiac work involves the observation that the HR was progressively reduced with continuous flow LVAD unloading. This reduction in HR is unlikely to be mediated by autonomic reflexes as neither atropine or β1 adrenergic blockade altered this progressive bradycardia (FIG. 3C). Furthermore, changes in left atrial pressures (decrease) and loss of pulsatility within the aorta should produce a reflex tachycardia even in light of normal venous pressures and preserved MAP. The effect of the decreased HR on TP was in line with known changes in SW and MVO₂ during LVAD unloading. Therefore, and perhaps because of continuous flow LV unloading, changes in HR contribute meaningfully to the observation that the TP correlated well with changes in stroke work and the TPIEDP was linear in almost all cases.

### Linearity of the MTP

The fact that TP was linearly related to changes in LVEDP is an important issue with regards to the utility of the TPIEDP for the assessment cardiac function. The Frank-Starling (F-S) relationship is known to be curvilinear—with a plateau evident at higher filling pressures. Glower and colleagues demonstrated, as Sarnoff and BergIund hypothesized, that substituting EDV for EDP would make the F-S relationship linear. TP/EDP was found to be linear over the full range of LV volumes studied with the exception of PE infusion after autonomic blockade, where a plateau was observed at the highest filling pressures. However, the M_{TP} varied little between days and doses of phenylephrine below this plateau—or below the so-called “flex point”. It is believed that the experimental conditions of increased afterload combined with autonomic blockade in this single animal mimic the expected results in experimental or clinical heart failure.

The possibility exists that a plateau in the MTP would not routinely be observed in clinical heart failure cases. In all the sheep studied, the LVEDP was elevated upon reloading of the LV prior to a ‘run’ (before esmolol Δ 5.0±1.6 mm Hg and after esmolol Δ 11.5±1.9 mm Hg), a level of acute volume loading that should theoretically be sufficient to reveal a plateau. However, in all animals studied, no plateau was observed even after β1 blockade (esmolol). Another explanation for the lack of an observed plateau in all studies is that the approximate 1 L/min of flow (@ 6,000 rpm) still present upon LV reloading was sufficient to prevent the observation of a plateau, i.e., prevented full reloading of the left ventricle.

However, based on the animals studied, another likely explanation for the preserved linearity of the TP/EDP, and again a condition specific to the method of LVAD unloading, is that changes in LVEDP during LVAD unloading were relatively small per unit time in the face of preserved right sided and systemic hemodynamics (Table 1, FIG. 6). Acute right ventricular collapse causing septal bulging during vena cava occlusion has been posited as an explanation for the nonlinearity of the F-S relationship reliant on LVEDP [Olsen C O. Tyson G S. Maier G W, Spratt J A. Davis J W. Rankin J S. Dynamic ventricular interaction in the conscious dog. Circ Res. 1983: 52: 85. and Glower]—an event that would not be applicable during acute LYAD unloading. Thus, the small incremental changes in LVEDP in the face of supported right ventricular pressures and supported pericardial pressures [Tyson G S, Maier G W. Olsen C O. Davis J W. Rankin J S. Pericardial influences on ventricular filling in the conscious dog: an analysis based on pericardial pressure. Circ Res. 1984: 54: 173.] during a ‘run’ likely allowed the LV ‘VP to linearly reflect changes in LVEDP or to remain better coupled with changes in LV end-diastolic pressure. Furthermore, it is believed that this coupling holds true for clinical cases of heart failure.

Irregardless of afterload sensitivity or reflex activation, if the TP/EDP was observed to be curvilinear in clinical heart failure cases, then the point where TP assumes a linear relationship to EDP, ‘flex point’, maybe of additional diagnostic and prognostic importance. The ‘flex point’ could hypothetically be a target for support—e.g. 75% of flex. Furthermore, the ‘flex point’ would be data not traditionally available from the P-V relationship, and additional study would be needed if plateauing of the MTP proves in the future to be clinically or experimentally evident.

### Reflex Activation

Among potential confounding issues related to LV unloading is autonomic reflex activation. Foremost among these would be the impact to the right sided and systemic hemodynamics. The right ventricular hemodynamics were unaltered during LVAD unloading ‘runs’ (Table 1, FIG. 6). Therefore, it is unlikely that altered venous filling pressures would have contributed in any substantial way to alter autonomic tone in any particular direction. However, the potential for alteration in arterial and left atrial hemodynamics during unloading ‘run’ still exists.

Left atrial baroreflex activation (Bainbridge reflex) upon reloading of the atria could have affected the TP/EDP relationship. Increased sympathetic drive was evident upon reloading of the LV as both esmolol (N=6 animals) and complete autonomic blockade (N=3 days) reduced, though not significantly (power=0.45), the observed increase in HR (Tables I and 2). Additionally, vagal withdrawal, also associated with the Bainbridge reflex, was nearly complete given that atropine administration (N=3 days) did not further increase heart rate above that of LV reloading alone. So it would seem likely that reflex activation and then its subsequent withdrawal—during LVAD unloading—could have affected the TP/EDP. The only evidence contrary to this or that would support a minimal role for the impact of reflex activation is from a comparison reported of the PRSW obtained during an inferior vena cava occlusion and later during a LVAD unloading ‘run’. Theoretically, the vena cava occlusion would be completed prior to reflex activation (<10 seconds). No difference was observed in the M_{w} or its intercept based on the method of LV unloading. Most likely it is probably not hemodynamically valid to compare the TP/EDP during a vena cava occlusion with that obtained during a LVAD ‘run’ because, as described earlier, of the reliance of the dP/dtₘₐₓ on the LV developed pressure. This further illustrates that the validity of the TP/EDP would likely not be applicable to all situations of LV unloading—preload should vary independent of afterload for the TPJEDP to be meaningful.

Lowered arterial systolic and pulse pressures, even in the face of maintained MAP, are known to reflexively increase sympathetic drive and mediate vagal withdrawal leading to increase inotropy and heart rate, respectively and concomitantly. However, it is unlikely that the modest changes in aortic systolic pressure would be a primary stimulus to activate aortic baroreceptors, but the loss of aortic pulsatility may have been sufficient to also increase sympathetic efferent tone upon LV reloading (Bainbridge reflex).

The relationship of TP to EDP (TP/EDP), derived solely from the LV pressure signal in sheep partially supported with a continuous flow LVAD (HeartMate II®), establishes a proof of concept methodology for the assessment of LV function in patients supported with an LVAD. The use of an axial flow LVAD to acutely reload and then unload the LV (‘run’) for the purposes of establishing preload recruitable function was reliable and reproducible. The LV triple product correlated with stroke work during LVAD ‘runs’. Also, changes observed in the TP/EDP relationship (slope: M_{TP}) were similar to those observed in the PRSW and reflect alterations in cardiac inotropy in LVAD supported sheep. Preliminary data demonstrated that the M_{Tp} was independent of physiological conditions of increased afterload. Though the method for assessment of TP/EDP as detailed here is likely only amenable to continuous flow LVADs, left ventricular pressure data should prove valuable in all patients supported with mechanical circulatory support—especially in those instances where criteria for and the potential to ‘wean’ are critical: e.g. post-cardiotomy cardiogenic shock, pregnancy-associated cardiogenic shock and acute myocarditis.

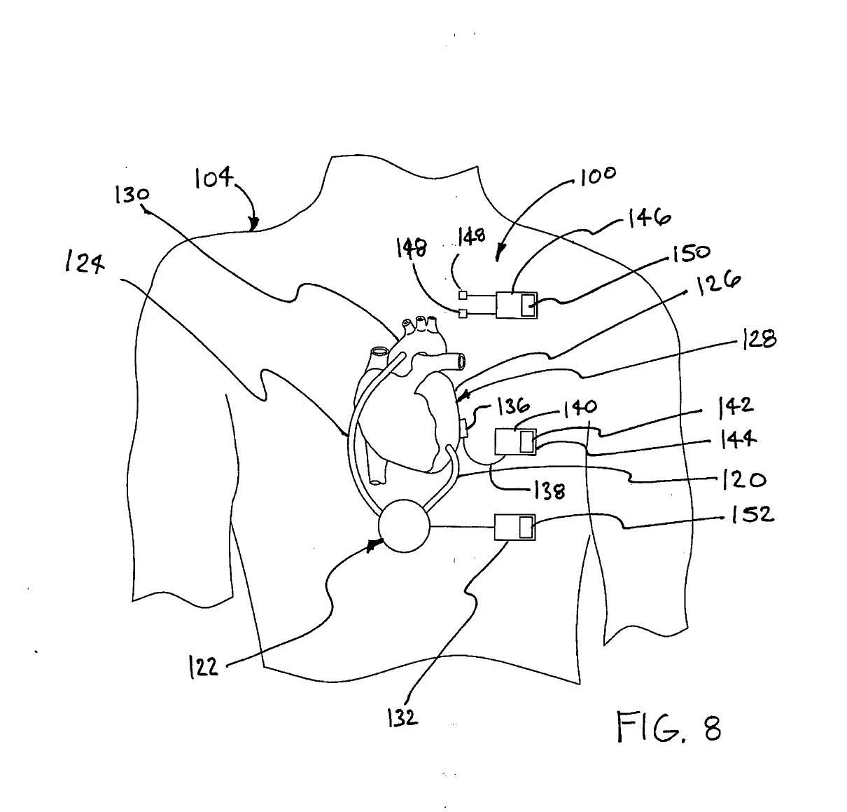

FIG. 8 is a schematic diagram illustrating a system 100 in accordance with one exemplary embodiment of the present invention. System 100 includes a blood pump 102 that is implanted in a human body 104. Blood pump 102 includes an inflow cannula 120, a pump housing 122, and an outflow cannula 124. Inflow cannula 120 attaches to an inlet side of pump housing 122 and extends through the wall of a left ventricle 126 of a heart 128. Outflow cannula 124 attaches to an outlet side of pump housing 122 and extends through a wall of an aorta 130 of human body 104. Blood pump 102 may comprise various blood pumps without deviating from the spirit and scope of the present invention. Blood pumps that may be suitable in some applications are commercially available from, for example, Thoratec Corporation of Pleasanton, Calif.

System 100 of FIG. 8 includes a controller 132 that is operatively coupled to blood pump 102. System 100 also includes a pressure measurement device 134. In the embodiment of FIG. 8, pressure measurement device 134 is capable of providing left ventricular pressure values to controller 132. Pressure measurement device 134 includes a remote sensor assembly 136 for measuring endocardial pressure that is connected via a lead 138 to at telemetry unit 140 for telemetering measured pressure data.

Pressure measurement devices that may be suitable in some applications are disclosed in U.S. Pat. Nos. 6,033,366; 6,296,615; 6,379,308; 6,409,674; 6,659,959; 7,025,727 and United States Patent Application numbers 2002/0120200; 2005/0182330. The entire disclosure of each of the above-mentioned United States patents and patent applications is hereby incorporated by reference herein.

Lead 138 connects remote sensor assembly 136 to telemetry unit 140. Lead 138 may contain, for example, four conductors—one each for power, ground, control in, and data out. Lead 138 may incorporate conventional lead design aspects as used in the field of pacing and implantable defibrillator leads. Lead 138 may also include a connector that allows remote sensor assembly 136 to be connected and disconnected from the telemetry unit 140 in the surgical suite to facilitate ease of implantation, at a later time should it be necessary to change the telemetry unit 140, or for any other circumstance.

Telemetry unit 140 includes telemetry electronics 142 contained within a housing 144. Housing 144 protects the telemetry electronics from the harsh environment of the human body. Housing 144 may be fabricated of a suitable biocompatible material such as titanium or ceramic and is hermetically sealed.

System 100 of FIG. 8 also includes a heart rate sensor 146. Heart rate sensor 146 may be capable of, for example, providing heart rate values to controller 132. In the embodiment of FIG. 8, heart rate sensor comprises a plurality of ECG electrodes 148. In the embodiment of FIG. 8, ECG electrodes 148 are shown making electrical contact with human body 104. ECG electrodes 148 may be placed on the surface of the skin of human body 104 or implanted underneath the skin of human body 104. Although two ECG electrodes 148 are illustrated in the embodiment of FIG. 8, it will be appreciated that more or fewer ECG electrodes 148 may be utilized without deviating from the spirit or scope of the present invention.

Heart rate sensor 146 is capable of collecting an ECG signal representative of a patient's cardiac rhythm. Heart rate sensor 146 is also capable of deriving a heart rate from that ECG signal. In the embodiment of FIG. 8, heart rate sensor 146 includes a wireless communication circuit 150 that is capable of for telemetering heart rate values. Controller 132 of FIG. 8 includes telemetry circuitry 152. Telemetry circuitry 152 enables controller 132 to receive heart rate values from heart rate sensor 146. Telemetry circuitry 152 also enables controller 132 to receive left ventricular pressure values from pressure measurement device 134.

In the embodiment of FIG. 8, controller 132 uses heart rate values received from heart rate sensor 146 and ventricular pressure values received from pressure measurement device 134 to calculate an estimated cardiac work. Controller 132 may then deliver control signals to blood pump 102 in response to the estimated cardiac work. Controller 132 may comprise various elements without deviating from the spirit and scope of the present invention. For example, controller 132 may comprise a microprocessor. By way of a second example, controller 132 may comprise an application specific integrated circuit (ASIC) comprising a plurality of logic gates.

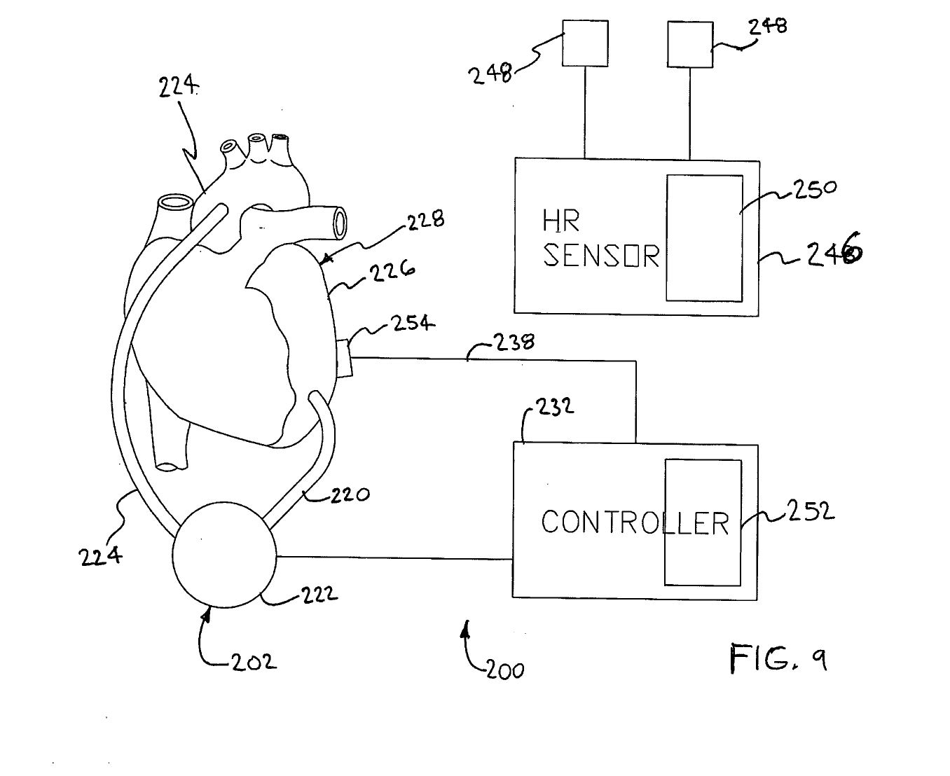

FIG. 9 is a schematic diagram showing a system 200 in accordance with an additional exemplary embodiment of the present invention. System 200 includes a blood pump 202 comprising an inflow cannula 220, a pump housing 222, and an outflow cannula 224. In the embodiment of FIG. 9, inflow cannula 220 is shown extending through the wall of a left ventricle 226 of a heart 228. Also in the embodiment of FIG. 9, outflow cannula 224 is shown extending through the wall of an aorta 230. With reference to FIG. 9, it swill be appreciated that blood pump 202 establishes a blood flow path between left ventricle 226 and aorta 230.

System 200 of FIG. 9 includes a controller 232 that is operatively coupled to blood pump 202. A pressure sensor 254 of system 200 is electrically connected to controller 232 by a lead 238. In the embodiment of FIG. 9, pressure sensor 254 is capable of providing left ventricular pressure values to controller 232. Lead 238 may contain, for example, four conductors—one each for power, ground, control in, and data out. Lead 238 may incorporate conventional lead design aspects as used in the field of pacing and implantable defibrillator leads. Lead 238 may also include a connector that allows pressure sensor 254 to be connected and disconnected from controller 232 in the surgical suite to facilitate ease of implantation, at a later time should it be necessary to change pressure sensor 254, or for any other circumstance.

System 200 of FIG. 9 also includes a heart rate sensor 246. Heart rate sensor 246 is capable of collecting an ECG signal representative of a patient's cardiac rhythm. Heart rate sensor 246 is also capable of deriving a heart rate from the ECG signal. In the embodiment of FIG. 9, heart rate sensor 246 includes a wireless communication circuit 250 that is capable of for telemetering heart rate values. Controller 232 of FIG. 9 includes telemetry circuitry 252. Telemetry circuitry 252 enables controller 232 to receive heart rate values from heart rate sensor 246. In the embodiment of FIG. 9, controller 232 uses heart rate values received from heart rate sensor 246 and ventricular pressure values received from pressure sensor 254 to calculate an estimated cardiac work. Controller 232 may then deliver control signals to blood pump 202 in response to the estimated cardiac work.

In the embodiment of FIG. 9, heart rate sensor comprises a plurality of ECG electrodes 248. ECG electrodes 248 may be placed on the surface of the skin of a human body and/or implanted underneath the skin of a human body. Although two ECG electrodes 248 are illustrated in the embodiment of FIG. 9, it will be appreciated that more or fewer ECG electrodes 248 may be utilized without deviating from the spirit or scope of the present invention.

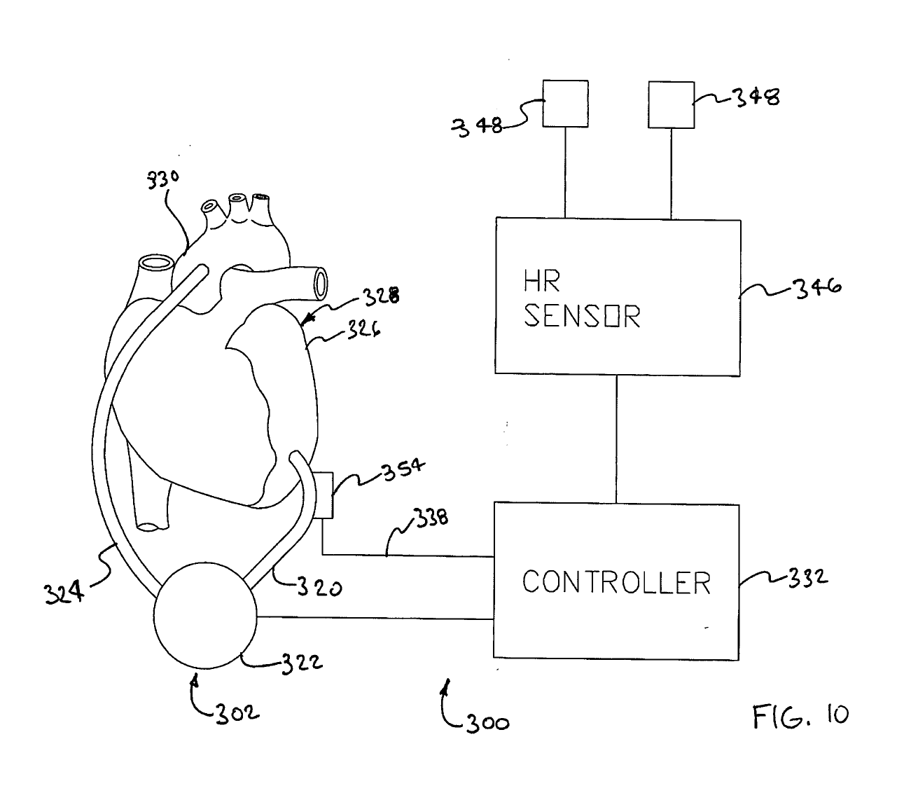

FIG. 10 is a schematic diagram showing a system 300 in accordance with another exemplary embodiment of the present invention. System 300 includes a blood pump 302 comprising an inflow cannula 320, a pump housing 322, and an outflow cannula 324. With reference to FIG. 10, it will be appreciated that blood pump 302 establishes a blood flow path between the left ventricle 326 of a heart 328 and an aorta 330. Blood pump 302 may comprise various blood pumps without deviating from the spirit and scope of the present invention. Blood pumps that may be suitable in some applications are commercially available from Thoratec Corporation of Pleasanton, Calif.

System 300 of FIG. 10 includes a controller 332 that is operatively coupled to blood pump 302. A pressure sensor 354 of system 300 is electrically connected to controller 332 by a lead 338. In the embodiment of FIG. 10, pressure sensor 354 is coupled to inflow cannula 320 so that pressure sensor 354 is capable of providing pump inlet pressure values to controller 332. In the embodiment of FIG. 10, inflow cannula 320 is shown extending through the wall of the left ventricle 326 of heart 328. Also in the embodiment of FIG. 10, outflow cannula 324 is shown extending through the wall of aorta 330.

System 300 of FIG. 10 also includes a heart rate sensor 346. In the embodiment of FIG. 10, heart rate sensor 346 is connected to controller 332 by a lead so that heart rate sensor 346 can provide heart rate values to controller 332. Heart rate sensor 346 is capable of collecting an ECG signal representative of a patient's cardiac rhythm. Heart rate sensor 346 is also capable of deriving a heart rate from the ECG signal. In, the embodiment of FIG. 10, heart rate sensor comprises a plurality of ECG electrodes 348. ECG electrodes 348 may be placed on the surface of the skin of a human body and/or implanted underneath the skin of a human body.

In the embodiment of FIG. 10, controller 332 uses heart rate values received from heart rate sensor 346 and pump inlet pressure values received from pressure sensor 354 to calculate an estimated cardiac work. Controller 332 may then deliver control signals to blood pump 302 in response to the estimated cardiac work. Controller 332 may comprise various elements without deviating from the spirit and scope of the present invention. For example, controller 332 may comprise a microprocessor. By way of a second example, controller 332 may comprise an application specific integrated circuit (ASIC) comprising a plurality of logic gates.

What is claimed as the invention is:

1. A method, comprising the steps of:
calculating an estimated cardiac work performed by a beating heart; and
varying the speed of a blood pump as a function of the estimated cardiac work.

2. The method of claim 1, wherein calculating the estimated cardiac work comprises:
detecting a heart rate (HR) of the beating heart;
monitoring left ventricular (LV) pressure in the beating heart;
calculating a maximum derivative of the LV pressure (dp/dt max);
determining a left ventricular systolic pressure (LVSP) from the monitored LV pressure; and
calculating a triple product (TP) defined by the equation:
TP=dp/dt max*HR*LVSP.

3. The method of claim 2, further comprising:
determining a left ventricular end diastolic pressure (EDP) from the monitored LV pressure; and
calculating a ratio (M) defined by the equation: M=TP/EDP.

4. The method of claim 1, wherein varying the speed of the blood pump comprises varying an electrical signal provided to the blood pump by a controller.

5. The method of claim 4, wherein varying the electrical signal provided to the blood pump by the controller comprises varying a current of the electrical signal.

6. The method of claim 4, wherein varying the electrical signal provided to the blood pump by the controller comprises varying a voltage of the electrical signal.

7. The method of claim 4, wherein varying the electrical signal provided to the blood pump by the controller comprises varying a pulse rate of the electrical signal.

8. The method of claim 4, wherein varying the electrical signal provided to the blood pump by the controller comprises varying a pulse duration of the electrical signal.

9. The method of claim 1, wherein the variation in the speed of the blood pump is inversely proportional to the estimated cardiac work.

10. A method, comprising the steps of:
detecting a heart rate (HR) of a beating heart;
monitoring left ventricular (LV) pressure in the beating heart;
calculating a maximum derivative of the LV pressure (dp/dt max);
determining a left ventricular systolic pressure (LVSP);
calculating a triple product (TP) defined by the equation:
TP=dp/dt max*HR*LVSP;
and
varying the speed of a blood pump as a function of TP.

11. The method of claim 9, further including pumping blood from a left ventricle of the heart to an aorta.

12. The method of claim 9, wherein detecting heart rate comprises detecting an electrocardiogram signal.

13. The method of claim 9, wherein the variation in the speed of the blood pump is inversely proportional to TP.

14. The method of claim 9, wherein varying the speed of the blood pump comprises varying an electrical signal provided to the blood pump by a controller.

15. The method of claim 9, further comprising providing left ventricular pressure values to a controller via wireless transmission.

16. The method of claim 9, further comprising providing heart rate values to a controller via wireless transmission.

17. A method, comprising the steps of:
detecting a heart rate (HR) of a beating heart;
monitoring left ventricular (LV) pressure in the beating heart;
calculating a maximum derivative of the LV pressure (dp/dt max);
determining a left ventricular systolic pressure (LVSP);
calculating a triple product (TP) defined by the equation:
TP=dp/dt max*HR*LVSP;
and
determining a left ventricular end diastolic pressure (EDP) from the monitored LV pressure; and
calculating a ratio: (M) defined by the equation: M=TP/EDP;
varying the speed of a blood pump as a function of M.

18. An apparatus, comprising:
a blood pump;
a controller operatively coupled to the blood pump;
a pressure sensor providing left ventricular (LV) pressure values to the controller;
a heart rate sensor providing heart rate (HR) values to the controller; and
a controller operative to receive the sensed LV pressure values, receive the sensed HR values, calculate an estimated cardiac work, and deliver control signals to the blood pump in response to the estimated cardiac work.

19. The apparatus of claim 18, wherein the controller calculates a maximum derivative of the LV pressure (dp/dt max) from the LV pressure values.

20. The apparatus of claim 18, wherein the controller derives a left ventricular systolic pressure (LVSP) from the LV pressure values.

21. The apparatus of claim 18, wherein the controller calculates a triple product (TP) defined by the equation:
TP=dp/dt max*HR*LVSP.

22. The apparatus of claim 21, wherein the controller calculates the estimated cardiac work as a function of TP.

23. The apparatus of claim 21, wherein the controller varies a speed of the blood pump as a function of TP.

24. The apparatus of claim 23, wherein the variation in the speed of the blood pump is inversely proportional to TP.

25. The apparatus of claim 21, wherein the controller calculates a ratio M defined by the equation: M=TP/EDP;
where EDP is an end diastolic pressure derived from the LV pressure values.

26. The apparatus of claim 18, wherein the blood pump comprises an inlet cannula dimensioned to be inserted through a wall of a left ventricle of the heart.

27. The apparatus of claim 26, wherein the blood pump comprises an outlet cannula dimensioned to be inserted through a wall of an aorta.

28. The apparatus of claim 27, wherein the blood pump defines a blood flow path between the left ventricle and the aorta.

29. The apparatus of claim 18, wherein the controller comprises a power supply that provides an electrical signal to the blood pump.

30. The apparatus of claim 18, wherein the controller comprises a regulator that regulates the flow of a fluid to the blood pump.

31. The apparatus of claim 30, wherein the fluid comprises a gas.

32. The apparatus of claim 31, wherein the fluid comprises a liquid.

33. The apparatus of claim 18, wherein the controller comprises an application specific integrated circuit (ASIC) comprising a plurality of logic gates.

34. The apparatus of claim 18, wherein the controller comprises a microprocessor.

35. The apparatus of claim 18, further comprising a wireless communication circuit connected to the pressure sensor for providing left ventricular pressure values to the controller via wireless transmission.

36. The apparatus of claim 18, further comprising a wireless communication circuit connected to the heart rate sensor for providing heart rate values to the controller via wireless transmission.

37. A method for determination of cardiac function by monitoring left ventricular (LV) pressure and varying ventricular assist device (VAD) speed, utilizes a relationship of the end-diastolic LV pressure (LVEDP) to an estimate of LV cardiac work calculated from the LV pressure signal by the triple product (TP): dp/dtmax*HR*LVSP, wherein the slope of a regression analysis of the comparison of TP vs LVEDP is used as an indicator of a patient's cardiac efficiency and analogous and comparable to preload recruitable stroke cardiac work as calculated from direct volume measurement of the LV, and of native cardiac function of a patient supported by a VAD.
